(19) United States
(12) Patent Application Publication
(10) Pub. No.: US 2014/0273258 A1
(43) Pub. Date: Sep. 18, 2014

(54) Functional Element for Arranging in Front of the Active Measuring Region of a Sensor Element

(71) Applicant: Robert Bosch GmbH, Stuttgart (DE)

(72) Inventors: Richard Fix, Gerlingen (DE); Andreas Krauss, Tuebingen (DE)

(73) Assignee: Robert Bosch GmbH, Stuttgart (DE)

(21) Appl. No.: 14/198,024

(22) Filed: Mar. 5, 2014

(30) Foreign Application Priority Data
Mar. 12, 2013 (DE) 10 2013 204 262.0

(51) Int. Cl. G01N 33/00

(52) U.S. Cl. CPC G01N 33/0024
USPC 436/111; 422/93; 436/116; 436/143

(57) ABSTRACT

A functional element positioned in front of an active detection region of a sensor element has a compact main body with a gas-side surface, a sensor-side surface, and at least two functional channels located between the gas-side surface and the sensor-side surface. The functional channels have a functionality that is different from one another with respect to a gas that can pass through. Such a functional element allows particularly variable operation of a sensor with great long-term stability.

This application claims priority under 35 U.S.C. §119 to patent application no. DE 10 2013 204 262.0, filed on Mar. 12, 2013 in Germany, the disclosure of which is incorporated herein by reference in its entirety.

The present disclosure relates to a functional element for arranging in front of the active measuring region of a sensor element. The present disclosure also relates to a sensor arrangement having such a functional element and a sensor element.

### BACKGROUND

Sensors or sensor arrangements are known in many applications and are widely used. Among the known sensors there are for example gas sensors, which can qualitatively and/or quantitatively investigate a gas or gas mixture to be investigated for its constituents.

In the case of present-day sensors, such as for example exhaust gas sensors, the sensor element, for example a ceramic sensor element, is exposed directly to the exhaust gas to be analyzed. It is also true for a newly developed generation of gas sensors, in the case of which the sensor element is configured as a miniature component in a very small installation space, that the sensor elements are likewise exposed directly to the surrounding gas or the gas stream to be measured. For example, a gas sensor or the sensor element may in this case be protected from large particles by a porous membrane, in order in this way to prevent mechanical damage to the sensor element.

### SUMMARY

The subject matter of the present disclosure is a functional element for arranging in front of the active detection region of a sensor element, having a compact main body with a gas-side surface and a sensor-side surface, at least two functional channels being arranged between the gas-side surface and the sensor-side surface, which functional channels have a functionality that is different from one another with respect to a gas that can pass through.

For the purposes of the present disclosure, a gas-side surface may be in particular a surface which faces in the direction of the gas to be measured when the functional element is arranged in front of the active measuring region of a sensor element. Correspondingly, a sensor-side surface of the main body may be in particular such a surface which is made to face in the direction of the sensor element when the functional element is arranged in front of the active measuring region of a sensor element.

Furthermore, for the purposes of the present disclosure, a functional channel may be a channel or a guide for a gas in which the gas passed through can be subjected in particular to a treatment as a result of it being possible for a functional element to be arranged, for which reason the channel may have at least one function with respect to the gas. For example, the function may however also be realizable without the provision of an explicit functional element, for instance in the provision of a reference value.

A pretreatment of the gas or a corresponding function may in this case mean any effect to which a gas is exposed, without being restricted in this respect in the scope of the disclosure.

For the purposes of the present disclosure, a gas to be measured may be understood as meaning in particular any gas, that is to say a gas comprising a substance or a gas comprising a number of substances, that is to say a gas mixture.

A functional element described above can make it possible in an advantageous way to operate an overall sensor by applying a plurality of different gas pretreatments.

For this purpose, the functional element has firstly a main body with a gas-side surface and a sensor-side surface. Consequently, the main body of the functional element comprises a surface or a side which can be arranged on the active measuring surface of a sensor element and a further surface which can be exposed to the gas to be measured. The active measuring surface of the sensor element is in this case in particular the surface by which the sensor element can come into contact with the gas to be measured and can thereby perform a measurement. Furthermore, the sensor element is in particular the active part of a sensor. Consequently, the functional element serves for at least partially covering, in particular completely covering, the active surface of the sensor element, and in this case for being arranged upstream of the sensor surface with respect to the gas to be measured. In other words, a gas to be measured flows through the functional element before it is detected at the sensor element or at the active measuring surface of the sensor element.

To make this possible, at least two different functional channels are arranged between the gas-side surface and the sensor-side surface. The functional channels may in this case represent in particular the only connections between the gas-side surface and the sensor-side surface of the functional element, since the main body of the latter may be compact, and consequently substantially gas-impermeable. Furthermore, the functional channels may in particular be different with respect to their function. For example, different pretreatments may be performed.

The functional channels may in this case serve by way of example for pretreating the gas prior to detection at the active measuring surface of the sensor element. Consequently, it may be possible by a functional element described above not only to make a defined pretreatment possible, but also to make a plurality of different gas pretreatments possible for an overall sensor.

In this case, the number of functional channels, and consequently likewise the number of pretreatment steps that can be carried out in parallel, for a gas to be measured may be substantially freely selectable. For example, such a functional element may be provided with a plurality of functional channels, of which only some are assigned a function by the insertion of corresponding components. Further functional channels may for example make it possible to perform a measurement without a pretreatment of the gas to be measured as a reference value, and/or serve as reserve channels, which can be retrofitted at any time with a specific function. This can make particularly great variability possible, not only directly in the production of the functional elements but also throughout the entire operation or the entire lifetime of the functional element or of a sensor provided with it.

This may make it possible for example to measure differently pretreated gas species simultaneously. That makes it possible on the one hand to obtain a particularly high degree of selectivity, and on the other hand to be able to realize multi-gas sensors for gas mixtures.

Consequently, a functional element described above makes it possible in particular to carry out a plurality of parallel and different measuring methods using only one sensor element. This allows a compact sensor to be used to collect a plurality of items of information, for which it has often been necessary according to the prior art to use a plurality of sensors that had to be arranged separately from one another. By contrast with these solutions from the prior art, such a diversity of measurements can be made possible by a functional element described above, even with a compact installation space.

However, not only is a wide diversity of measurements made possible here, but also such a diversity of measurements is achieved in a particularly short time, since the measurements can be carried out not one after the other but rather substantially in parallel in terms of time.

The aforementioned advantages can be achieved here by using a single component, which can in particular be produced at low cost and can be combined with known sensor elements in an easy way. Consequently, the functional element described above can also be integrated in an easy way into existing sensors, which also makes the retrofitting of existing sensors particularly easy.

To sum up, such a functional element makes significant advantages possible here for the sensor function and stability of a sensor element by allowing in principle any number of pretreatments of the gas that is to be measured to be carried out substantially in parallel, and consequently also a plurality of measuring methods to be carried out.

Within the scope of one configuration, the main body may be at least partially made of a material that is selected from the group consisting of silicon, silicon carbide, silica, alumina, semiconductors and glass. Advantageous in particular are materials that can be structured well by means of methods of microsystems technology, for example semiconductor substrates such as Si wafers or glass substrates such as Foturan. For example, the main body may be made completely of such a material, or consist of such a material. The use of an aforementioned material may make it possible in particular to work or structure it by means of methods of microsystems technology that are known per se and fully developed. Known here for example are methods and processes for producing defined holes, chambers, or porous regions. This applies in particular to silicon or silicon-based materials. Similarly, there are known methods for the processing of electrical connections or heating elements, which may likewise be of advantage, as explained later in detail. The use of methods of microsystems technology may be advantageous here, since on the one hand even sensor elements of small dimensions can be fitted with a functional element, or on the other hand it may also be possible for functional elements to be produced with extremely small dimensions. For example, a sensitive region of a gas sensor located under the microstructured pretreatment device, such as for instance a chip of the size 2×2 mm², may have a surface area of 10*100 μm² and be located a few hundreds of μm away from a second gas-sensitive region. With methods of microsystems technology, it is possible to adapt the microstructured pretreatment device precisely to these area sizes and distances of the sensitive regions of the chip, and also furthermore to make the edge angles of the openings defined, for example at a 45° angle. In addition, with such methods, high-precision structuring can be performed, which may be of advantage in particular in the case of high-precision measurements. Furthermore, the aforementioned methods of microsystems technology allow in particular very low-cost production of such a functional element.

Within the scope of a further configuration, at least one functional channel may be at least partially temperature-controllable, that is to say at least to a locally limited extent. This configuration may be of advantage for many applications or functions. For example, temperature control of a functional channel allows the gas flowing through to be cooled before it impinges on the sensor element, so that damage to the sensor element due to excessively high temperatures of the gas to be measured can be prevented. Furthermore, a constant temperature of the gas to be measured may be of advantage for particularly precise measurement. In addition, a multiplicity of pretreatment steps for a gas to be measured can proceed particularly advantageously at an elevated temperature or in principle under defined temperature conditions. Consequently, a gas pretreatment can proceed in a particularly preferred way in a temperature-controlled functional channel. It may in this case be of advantage for example for the forming of a temperature-controlled channel if, as described above, the main body is formed from a material that can be processed by methods of microsystems technology. This is so because, with such materials or with such methods, temperature-controlling structures, such as for example cooling channels or heating channels of small dimensions, can be incorporated without any problem and at low cost.

Within the scope of a further configuration, at least one functional channel may have at least one functional channel element that is selected from the group consisting of a catalyst, a diffusion barrier, an ion-conducting material and a storage medium.

With respect to a catalyst, a reaction of the gas to be measured or a constituent of a gas mixture to be measured may be made possible for example. In particular, it may be made possible by catalytically active materials, such as for example platinum or activated carbon, to filter out certain substances from a gas mixture or to set thermodynamic equilibria of certain gas species. In principle, heatability of the functional channel or of individual functional regions within the functional channel may be of advantage in combination with a catalyst. Gas-permeable, such as for instance porous, materials arranged in the functional channels may be used for example as the carrier for such catalytically active materials, in order to maximize the surface area of the catalyst and consequently the catalyst conversion. For example, using a sensor with a functional element described above that has two functional channels, a gas mixture which comprises nitrogen monoxide along with nitrogen dioxide can be analyzed. In this case, a catalyst material that converts all of the nitrogen monoxide into nitrogen dioxide may be used in a functional region in one functional channel. In a further functional channel there may be no catalytically active material provided, so that here the mixture of nitrogen monoxide and nitrogen dioxide is detectable unchanged at the active measuring surface of the sensor. Subsequently, both the total amount of nitrogen monoxide and nitrogen dioxide and the constituent concentrations of nitrogen dioxide and nitrogen monoxide can be determined from the difference between the sensor signals. Furthermore, a catalyst may be used to change the gas composition, so that for example certain gases are transformed in such a way that they can be detected particularly sensitively by the corresponding sensor elements.

A diffusion barrier may also be understood as meaning in particular such an element that can hinder or set in a defined manner the diffusion of a fluid medium, in particular a gas or a liquid. As an element given by way of example, a diffusion barrier may be understood as meaning a porous medium or a grid. By the provision of such diffusion barriers, it may be possible to influence the spread of changes in concentration. In the combination given by way of example of a functional channel with a high diffusion rate, for instance with a porous material having a comparatively great pore width, and a further functional channel with a low diffusion rate, for instance with a porous material having a comparatively small pore width, the sensor arranged on the functional element can differentiate the rate of changes in gas concentration in an advantageous way. Further advantages of diffusion barriers, such as for example porous media, are constituted by particularly good and secure protection of the sensor from abrasion, from direct water impact or particle impact and from soiling, such as for example from soot.

With respect to the ion-conducting materials, proton conductors or oxygen-ion conductors are known for example. If for instance an oxygen-ion conductor is used in a functional channel of the functional element, the signal of the sensor element or region of the sensor element located thereunder may be attributable exclusively to oxygen. Consequently, for example in the case of an oxygen-sensitive chemical sensor element or measuring principle, the oxygen sensitivity can be included in the comparison with a further functional channel by way of the signal processing. Examples of ion-conducting elements are Nernst cells or pumping cells known per se, which are configured as oxygen-ion conductors for example by using yttria-doped zirconia.

With respect to a storage medium, such as for example a getter, it may in particular have the effect that certain gas constituents are bound to the storage medium, so that they do not reach the sensor arranged downstream. When a selective storage material, such as for example a selective getter, is used in a functional channel, it is possible by comparison of this chemical sensor signal with that of a further active measuring region in which no such storage medium is arranged upstream to determine precisely the amount of substance that is specifically captured in this getter. As a result, a diverse evaluation of the measurement may be possible. In addition, it may be made possible in a particularly easy way to remove effectively from the gas stream substances that may be contained in the gas to be measured and act for example as sensor poisons, and consequently reduce the effectiveness of the sensor element, before the stream reaches the sensor element. Storage materials given by way of example comprise ceria for oxygen storage or barium-containing compounds for nitrogen storage by barium nitrate formation.

Within the scope of a further configuration, a functional channel may have two functional channel elements arranged one behind the other. In this configuration, consequently, one functional channel element may consequently be arranged downstream of a further functional channel element in a functional channel. Consequently, two pretreatments of the gas can be carried out before the gas to be measured in the functional channel reaches the corresponding measuring region of the sensor element. For example, a storage medium may be provided, downstream of which an oxidation catalyst is provided. In this way, for example, catalyst poisons can be effectively intercepted. In this case, the individual functional regions in a functional channel may also be heated. In this case, depending on the size and thermal conductivity of the overall sensor, it may also be possible that different regions in a functional channel are heated to different temperatures, or the temperature is modulated. Thus, for example, different catalyst temperatures, and consequently different thermodynamic equilibria, can be set.

Within the scope of a further configuration, a functional layer may be provided, which functional layer is arranged at least partially on the gas-side surface of the functional element, that is to say at least to a locally limited extent. In this configuration, consequently, for example only the functional channels, certain regions of the functional element or the entire functional element may be covered with the further functional layer. This further outer functional layer may for example have a storage medium, such as for instance a getter, which can act as protection from poisoning with substances that are not to come into contact with the functional element or the sensor element. In principle, the functional layer may in this case undertake any function described above with respect to the functional channels.

With regard to further advantages and features of the functional element according to the disclosure, reference is hereby made explicitly to the explanations in connection with the sensor arrangement according to the disclosure, the use according to the disclosure and the figures. Features and advantages according to the disclosure of the functional element according to the disclosure are also intended to be applicable to, and considered to be disclosed for, the sensor arrangement according to the disclosure and the use according to the disclosure, and vice versa. The disclosure also includes all combinations of at least two of the features that are disclosed in the description, the claims and/or in the figures.

The subject matter of the present disclosure is also a sensor arrangement, having a sensor element with an active detection surface and having at least one functional element configured as described above, the functional element being arranged upstream of the detection surface of the sensor element with respect to the direction of flow of the gas to be measured. In particular, such a sensor arrangement allows particularly secure measurement, a multiplicity of evaluations of the result in situ also being possible. In this case, the sensor arrangement may also operate particularly stably over a long time, since the sensor element can be protected particularly effectively from external influences.

Such a sensor arrangement consequently comprises firstly a functional element, which is configured as described above, for which reason reference is made in this respect to the statements made above concerning the functional element.

Furthermore, such a sensor arrangement comprises a sensor element that may be based on a solid electrolyte with ion-conducting properties or a chemically sensitive field-effect transistor. In principle, any sensor element or any measuring principle may be used, if the target application allows suitable conditions for the respective measuring principle. The sensor element may for example be a chemically sensitive field-effect transistor and in principle be a field effect-based gas sensor on the basis of the materials that are given by way of example, and are not restrictive, silicon (Si), silicon carbide (SiC) or gallium nitride (GaN). For example, the gas sensor may be a component such as that described for instance in the document DE 10 2007 003 541 A1, to which document reference is hereby expressly made. It may for example comprise a component that has a metallic layer on a substrate, the substrate being formed from a semiconductor material, and a diffusion barrier layer which is produced from a material that has a small diffusion coefficient for the material of the metallic layer being formed between the metallic layer and the substrate.

The provision of sensor elements that can be produced for example by semiconductor process technology also makes the integration of microelectronics possible, which is advantageous for example for the sensor signal conditioning. In particular for the simultaneous measurement of a number of possibly differently pretreated gas species that is possible according to the disclosure, it is advisable to use circuits for the signal processing. For example, the integrated microelectronics can be used to realize the following functions directly on the active measuring surface or on the sensor chip: signal amplification, signal filtering, analog/digital conversion, multiplexing, and consequently activation of a number of different sensors, linearization of a lambda step-change characteristic, offset calibration of the sensor characteristic, communication with sensor bus systems, etc.

The electronics of such a sensor system may also tap such signals that occur in the functional element, subject them to open-loop and closed-loop control and also use them for the signal processing of the sensor signals. These may be for example items of temperature information of the heating elements or currents or voltages of a potentially present Nernst cell or pumping cell.

Within the scope of one configuration, the functional element may be configured and fastened to the sensor element in such a way that each of the functional channels is connected to an independent locally limited detection region of a detection surface. In this configuration, consequently any functional channel may be assigned an independent region of the sensor element or an independent sensor element. Consequently, particularly precise measurements or particularly precise analyses of the measurement result may be possible. An independent and locally limited detection region may consequently be understood in particular as meaning a region on the surface of a sensor element that cannot interact with the detection regions of the further functional channels, or which surface is not fluidically connected to the surface of the further detection regions.

Within the scope of a further configuration, the functional element may be configured as a carrier for the sensor element. This may be possible for example in that the functional element has greater dimensions in a defined way than the sensor element. Furthermore, the functional element may be configured with suitable stability, which is sufficient to allow it to serve for the respective intended application as a carrier element. Consequently, apart from a protective function and pretreatment function, the functional element may also have a mechanical carrier function, it also being possible for the functional element to represent an electrical contacting function for the sensor element. This may be of advantage in particular whenever the production of the functional element is possible much more inexpensively than the production of a corresponding sensor element, for example on account of inexpensive materials, such as for example silicon for the functional element instead of silicon carbide for the sensor element, or if the corresponding production process is less expensive.

With regard to further advantages and features of the sensor arrangement according to the disclosure, reference is hereby made explicitly to the explanations in connection with the functional element according to the disclosure, the use according to the disclosure and the figures. Features and advantages according to the disclosure of the sensor arrangement according to the disclosure are also intended to be applicable to, and considered to be disclosed for, the functional element according to the disclosure and the use according to the disclosure, and vice versa. The disclosure also includes all combinations of at least two of the features that are disclosed in the description, the claims and/or in the figures.

The subject matter of the disclosure is also a use of a sensor arrangement configured as described above in a chemosensor, in particular in a gas sensor. In particular in the case of a gas sensor, a particularly precise or dynamic measurement result and also particularly variable evaluation of the measurement results can be obtained by a specific pretreatment of the gas to be measured prior to impingement on the detection surface of the sensor element, as described above.

Uses of gas sensors that are given here by way of example are lambda probes, nitrous oxide (NOx) sensors, hydrocarbon (HC) sensors, particle sensors, ammonia (NH3) sensors and further sensors for use for example in internal combustion engines, for example in the exhaust line, vehicles, and stationary installations, such as for example wood-burning stoves.

With respect to further chemosensors, in particular chemical sensors, there may be a use in consumer applications, such as for instance cell phones, household articles, gas warning systems, in medical equipment, such as for respiratory gas analysis, for instance in so-called lab-on-chip analytics, and for use in liquids, for instance for fuel analysis or for the analysis of body fluids.

With regard to further advantages and features of the use according to the disclosure, reference is hereby made explicitly to the explanations in connection with the sensor arrangement according to the disclosure, the functional element according to the disclosure and the figures. Features and advantages according to the disclosure of the use according to the disclosure are also intended to be applicable to, and considered to be disclosed for, the sensor arrangement according to the disclosure and the functional element according to the disclosure, and vice versa. The disclosure also includes all combinations of at least two of the features that are disclosed in the description, the claims and/or in the figures.

### BRIEF DESCRIPTION OF THE DRAWINGS

Further advantages and advantageous configurations of the subjects according to the disclosure are illustrated by the examples and drawings and are explained in the description that follows. It should be noted that the examples and drawings are only of a descriptive character and are not intended to restrict the disclosure in any form. In the drawings:

FIG. 1 shows a schematic view of an embodiment of a sensor arrangement according to the disclosure;

FIG. 2 shows a schematic view of a further embodiment of a sensor arrangement according to the disclosure; and

FIG. 3 shows a schematic view of a further embodiment of a sensor arrangement according to the disclosure.

### DETAILED DESCRIPTION

In FIG. 1, a schematic view of an embodiment of a sensor arrangement 10 according to the disclosure is shown. Such a sensor arrangement 10 may be used in particular in the case of a chemosensor, such as for example a gas sensor.

Such a sensor arrangement 10 comprises firstly a sensor element 12. In a way known per se, the sensor element 12 may for example be a chemically sensitive field-effect transistor and in principle be a field effect-based gas sensor on the basis of the materials that are given by way of example, and are not restrictive, silicon (Si), silicon carbide (SiC) or gallium nitride (GaN). In this case, the sensor element 12 has an active measuring surface or detection surface 14, which represents an active measuring region and may allow an investigation of a gas or gas mixture that is to be measured.

Furthermore, the sensor arrangement 10 has a functional element 16 for arranging in front of the active measuring region or the detection surface 14 of the sensor element 12. The functional element 16 comprises a compact main body 18 with a gas-side surface 20 and a sensor-side surface 22. The main body 18 may in this case be at least partially made of a material that is selected from the group consisting of silicon, silicon carbide, silica, alumina, semiconductors and glass. In this case, the functional element 16 is also arranged upstream of the detection surface 14 of the sensor element 12 with respect to the direction of flow of the gas to be measured.

The functional element 16 comprises at least two different functional channels 24 between the gas-side surface 20 and the sensor-side surface 22, in which different functional channels 24 a gas to be measured can be pretreated before reaching the sensor element 12. For example, at least one functional channel 24 may have at least one functional channel element 25 that is selected from the group consisting of a catalyst, a diffusion barrier, an ion-conducting material and a storage medium. In this case, it may also be provided that a functional channel 24 has two functional channel elements 25 arranged one behind the other. According to FIG. 1, two functional channels 24 have a diffusion barrier as a functional channel element 25. Furthermore, at least one functional channel 24 may be at least partially temperature-controllable.

It can also be seen in FIG. 1 that the functional element 16 is arranged upstream of the detection surface 14 of the sensor element 12 with respect to the direction of flow of the gas to be measured. The connection of the functional element 16 to the sensor element 12 may take place for example by a glass fusion 26, such as for instance by seal-glass bonding. It may be of advantage here that the glass fusion 26 takes place in a gastight manner, at least around the various gas-permeable functional channels 24, so that the pretreated gases can reach a defined detection region 28 of the sensor element 12 or of the detection surface 24 of the same. In other words, the functional element 14 is configured and fastened to the sensor element 12 in such a way that each of the functional channels 24 is connected to an independent locally limited detection region 28 of a detection surface 16. For a transmission of currents and voltages between the functional element 16 and the sensor element 12, contacts or electrical connections 40 may be present here between the sensor element 12 and the functional element 16.

In FIG. 2, a further configuration of a sensor arrangement 10 according to the disclosure is shown. The sensor arrangement 10 according to FIG. 2 corresponds in large parts to the sensor arrangement 10 from FIG. 1, so that the same or corresponding components are provided with corresponding reference signs and furthermore, for a more detailed description, reference can be made to the statements made concerning FIG. 1.

In the case of the configuration according to FIG. 2, it can also be seen that the functional element 16 is configured as a carrier for the sensor element 12. According to FIG. 2, this is possible by the functional element 16 having greater dimensions in a defined way than the sensor element 12, and consequently the functional element 16 being able to serve as a carrier structure for the sensor element 12.

In FIG. 3, a further configuration of a sensor arrangement 10 according to the disclosure is shown. The sensor arrangement 10 according to FIG. 3 corresponds in large parts to the sensor arrangement 10 from FIG. 1, so that the same or corresponding components are provided with corresponding reference signs and furthermore, for a more detailed description, reference can be made to the statements made concerning FIG. 1.

In the case of the configuration according to FIG. 3, it can also be seen that a functional layer 32 is provided, which functional layer 32 is arranged at least partially, in the configuration according to FIG. 3 completely, on the gas-side surface 20 of the functional element 14. In the configuration according to FIG. 3, furthermore, the sensor element 12 has greater dimensions than the functional element 14, and can consequently serve here, as in further configurations, as a carrier structure.

What is claimed is:

1. A functional element, comprising:
a main body that includes:
a gas-side surface; and
a sensor-side surface; and
at least two functional channels located between the gas-side surface and the sensor-side surface, wherein each of the at least two functional channels has a functionality that is different from one another with respect to a gas that can pass through;
wherein the functional element is configured to be positioned in front of an active detection region of a sensor element.

2. The functional element according to claim 1, wherein the main body comprises a material selected from the group consisting of silicon, silicon carbide, silica, alumina, semiconductors, and glass.

3. The function element according to claim 1, wherein at least one of the at least two functional channels is at least partially temperature controllable.

4. The functional element according to claim 1, wherein at least one of the at least two functional channels includes at least one functional channel element that is selected from a group consisting of a catalyst, a diffusion barrier, an ion-conducting material, and a storage medium.

5. The functional element according to claim 1, wherein at least one of the at least two functional channels includes a first functional channel element arranged behind a second functional channel element.

6. The functional element according to claim 1, further comprising a functional layer positioned at least partially on the gas-side surface.

7. A sensor arrangement, comprising:
a sensor element with an active detection surface; and
at least one functional element positioned upstream of the active detection surface with respect to a direction of flow, and comprising:
a main body that includes:
a gas-side surface; and
a sensor-side surface; and
at least two functional channels located between the gas-side surface and the sensor-side surface, wherein each of the at least two functional channels has a functionality that is different from one another with respect to a gas that can pass through;

8. The sensor arrangement according to claim 7, wherein the at least one functional element is configured and fastened to the sensor element such that each of the at least two functional channels is in communication with a respective independent locally limited detection region of the active detection surface.

9. The sensor arrangement according to claim 7, wherein the functional element is configured as a carrier for the sensor element.

10. A method of using a sensor arrangement in a chemosensor, comprising:
positioning the sensor arrangement in a flow of a gas to be measured;
passing the gas through each of at least two functional channels of a functional element, wherein:
the functional element comprises a main body that include a gas-side surface and a sensor-side surface such that the at least two functional channels are located between the gas-side surface and the sensor-side surface; and
each of the at least two functional channels has a functionality that is different from one another with respect to the gas; and
measuring the gas at respective independent locally limited detection region of an active detection surface of a sensor element corresponding to each of the at least two functional channels.

11. The functional element according to claim 1, wherein the main body is compact.

12. The sensor arrangement according to claim 7, wherein the main body is compact.

13. The method of using a sensor arrangement in a chemosensor according to claim 10, wherein the main body is compact.
